(19) United States
(12) Patent Application Publication
(10) Pub. No.: US 2012/0165640 A1
(43) Pub. Date: Jun. 28, 2012

(54) STRUCTURED BLOOD GLUCOSE TESTING PERFORMED ON HANDHELD DIABETES MANAGEMENT DEVICES

(75) Inventors: Paul J. Galley, Cumberland, IN (US); John F. Price, Mccordsville, IN (US)

(73) Assignee: ROCHE DIAGNOSTICS OPERATIONS, INC., Indianapolis, IN (US)

(21) Appl. No.: 12/977,531

(22) Filed: Dec. 23, 2010

(51) Int. Cl. A61B 5/145

(52) U.S. Cl. 600/365

(57) ABSTRACT

A handheld diabetes management device includes a bG measurement engine, a data store, a display, and a testing module. The bG measurement engine selectively measures bG levels in blood samples. The data store includes data for executing a plurality of structured tests, each of the structured tests calling for execution of one or more of: a first procedure including prompting the patient to input a first blood sample at a first predetermined time; a second procedure including prompting the patient to input second and third blood samples at second and third predetermined times, respectively; a third procedure including prompting the patient to input M blood samples at predetermined intervals beginning after a predetermined offset period; and a fourth procedure including prompting the patient to input N number of blood samples at N predetermined times of a day, respectively. The testing module selectively executes one or more of the structured tests.

### FIELD

The present disclosure relates to handheld medical devices and more particularly to handheld blood glucose (bG) management devices.

### BACKGROUND

Diabetes mellitus, often referred to as diabetes, is a chronic condition in which a person has elevated blood glucose levels that result from defects in the body's ability to produce and/or use insulin. There are three main types of diabetes. Type 1 diabetes usually strikes children and young adults, and can be autoimmune, genetic, and/or environmental. Type 2 diabetes accounts for 90-95% of diabetes cases and is linked to obesity and physical inactivity. Gestational diabetes is a form of glucose intolerance diagnosed during pregnancy and usually resolves spontaneously after delivery.

In 2009, according to the World Health Organization, at least 220 million people worldwide suffer from diabetes. In 2005, an estimated 1.1 million people died from diabetes. The incidence of diabetes is increasing rapidly, and it is estimated that between 2005 and 2030, the number of deaths from diabetes will double. In the United States, nearly 24 million Americans have diabetes with an estimated 25 percent of seniors age 60 and older being affected. The Centers for Disease Control and Prevention forecast that 1 in 3 Americans born after 2000 will develop diabetes during their lifetime. The National Diabetes Information Clearinghouse estimates that diabetes costs $132 billion in the United States alone every year. Without treatment, diabetes can lead to severe complications such as heart disease, stroke, blindness, kidney failure, amputations, and death related to pneumonia and flu.

Management of diabetes is complex because the level of blood glucose entering the bloodstream is dynamic. Variation of insulin in the bloodstream that controls the transport of glucose out of the bloodstream also complicates diabetes management. Blood glucose levels are sensitive to diet and exercise, but also can be affected by sleep, stress, smoking, travel, illness, menses, and other psychological and lifestyle factors that are unique to each patient. The dynamic nature of blood glucose and insulin, and all other factors affecting blood glucose, often require a person with diabetes to forecast blood glucose levels. Administration of insulin and/or oral medications can be regulated and timed to maintain blood glucose levels within an appropriate range at all times.

Management of diabetes is often highly intrusive because of the need to consistently obtain reliable diagnostic information, follow prescribed therapy, and manage lifestyle on a daily basis. Diagnostic information, such blood glucose level, can be obtained from a capillary blood sample with a lancing device and a test strip. The blood glucose level is measured via the test strip using a handheld blood glucose meter. Interstitial glucose levels can be obtained from a continuous glucose sensor worn on the body.

A therapy regimen for a patient can be established based on one or more of the patient's blood glucose levels. The therapy regimen can include administration of insulin and/or oral medication. Insulin can be administered with a syringe, an insulin pen, an ambulatory infusion pump, or a combination of two or more of the above. With insulin therapy, determining the amount of insulin to inject at a given time can require forecasting meal amount and composition (e.g., of fat, carbohydrates, and proteins, and amounts of each). Determining the amount of insulin to inject at a given time can also require consideration of the effects of exercise and physiologic state. The patient's management of lifestyle factors such as body weight, diet, and exercise can significantly influence the type and effectiveness of therapy.

Management of diabetes involves large amounts of diagnostic data and prescriptive data that are acquired from medical devices, personal health care devices, patient recorded information, health care professional tests results, prescribed medications and recorded information. Medical devices including self-monitoring bG meters, continuous glucose monitors, ambulatory insulin infusion pumps, diabetes analysis software, and diabetes device configuration software each of which generates or manages or both large amounts of diagnostic and prescriptive data. Personal health care devices can include weights, scales, blood pressure cuffs, pedometers, other activity monitors, and other suitable devices. Patient recorded data can include information relating to meals, exercise, and lifestyle. Health care professional biomarker data can include HbA1C, cholesterol, triglycerides, fasting glucose, and glucose tolerance. Health care professional recorded information can include therapy and other patient-specific information.

At the present time, a patient with diabetes can be asked by a health care professional to conduct, for example, a structured three day profile blood glucose (bG) test. A three day profile structured bG test involves the patient checking his or her bG level several times during each day for three days and hand writing the bG measurements in a chart. Preferably, the three day profile structured bG test is performed with the patient checking his or her bG level at seven different times on each of the three days and recording the seven different measurements each day. The seven different times at which a patient should measure and record his or her bG level are: 1) pre-breakfast; 2) post-breakfast; 3) pre-lunch; 4) post-lunch; 5) pre-dinner; 6) post-dinner; and 7) bedtime. The patient should consume breakfast at or shortly before measurements 1 and 2, lunch at or shortly before measurements 3 and 4, and dinner at or shortly before measurements 5 and 6. Based on the results of the three day profile structured bG test, a health care professional can determine or adjust an insulin therapy for the patient. There is a need for a handheld patient device to aggregate, manipulate, manage, present, and communicate diagnostic data and prescriptive data from medical devices, personal health care devices, patient recorded information, biomarker information and recorded information in an efficient manner. This would enable the patient to improve his or her care and health, to lead a full life, and to reduce the risk of complications from diabetes.

The background description provided herein is for the purpose of generally presenting the context of the disclosure. Work of the presently named inventors, to the extent it is described in this background section, as well as aspects of the description that cannot otherwise qualify as prior art at the time of filing, are neither expressly nor impliedly admitted as prior art against the present disclosure.

### SUMMARY

A handheld diabetes management device includes predetermined types of blood glucose (bG) data collection procedures for improved structured testing. The handheld diabetes management device includes a bG measurement engine, a data store, a display, and a testing module. The bG measurement engine selectively measures bG levels in blood samples of a patient and generates sample data indicative of the bG levels, respectively. The data store stores a first bG data collection procedure that prompts the patient to input at least one blood sample according to a first predetermined routine, a second bG data collection procedure that prompts the patient to input at least one blood sample according to a second predetermined routine that is different from the first predetermined routine, and a plurality of structured tests. Each of the structured tests is executable for determining a parameter related to diabetes care of the patient and has data for executing one or more of the first and second bG data collection procedures. Each of the first and second bG data collection procedures is accessible to each of the structured tests for execution. The testing module is in communication with the bG measurement engine, the display, and the memory. The testing module selectively executes one of the structured tests, including one or more of the first and second bG data collection procedures.

A handheld diabetes management device includes predetermined types of blood glucose (bG) data collection procedures for improved structured testing. The handheld diabetes management device includes a bG measurement engine, a data store, a display, and a testing module. The bG measurement engine selectively measures bG levels in blood samples of a patient and generates sample data indicative of the bG levels, respectively. The data store includes data for executing a plurality of structured tests, each of the structured tests calling for execution of one or more of: a first bG data collection procedure including prompting the patient to input a first blood sample at a first predetermined time; a second bG data collection procedure including prompting the patient to input second and third blood samples at second and third predetermined times, respectively; a third bG data collection procedure including prompting the patient to input M blood samples at predetermined intervals beginning after a predetermined offset period, wherein M is an integer greater than 2; and a fourth bG data collection procedure including prompting the patient to input N number of blood samples at N predetermined times of a day, respectively, wherein N is an integer greater than 6. The testing module is in communication with the bG measurement engine, the display, and the data store. The testing module selectively executes one of the structured tests including the one or more of the first, second, third, and fourth bG data collection procedures.

A handheld diabetes management device includes a blood glucose (bG) measurement engine that measures a bG level of a blood sample and further includes a touch screen display and a computer readable storage medium. The computer readable storage medium includes a non-modifiable portion and a modifiable portion. The non-modifiable portion includes firmware for operating the handheld diabetes management device and includes: a first routine executable for prompting the patient to input a first blood sample at a first predetermined time; a second routine executable for prompting the patient to input second and third blood samples at second and third predetermined times, respectively; a third routine executable for prompting the patient to input M blood samples at predetermined intervals beginning after a predetermined offset period, wherein M is an integer greater than 2; and a fourth routine executable for prompting the patient to input N number of blood samples at N predetermined times of a day, respectively, wherein N is an integer greater than 6. The modifiable portion includes data for executing one of a plurality of structured tests including: an order of execution of one or more of the first, second, third, and fourth routines for the one of the structured tests; entry, adherence, and exit criteria for each of the one or more of the first, second, third, and fourth routines; and at least one of a function and a mapping for determining a parameter related to diabetes care of the patient based on blood samples input pursuant to the one or more of the first, second, third, and fourth routines.

Further areas of applicability of the present disclosure will become apparent from the detailed description provided hereinafter. It should be understood that the detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings, wherein:

FIG. 1 shows a patient and a health care professional along with various devices that can be used to help the patient monitor and control health;

FIG. 2 shows a patient with a continuous glucose monitor (CGM), an ambulatory durable insulin infusion pump, an ambulatory non-durable insulin infusion pump, and a blood glucose (bG) management device;

FIG. 3 shows a diabetes care system of systems that can be used to manage diabetes;

FIG. 4 is a high level diagram of an example implementation of a handheld diabetes management device;

FIG. 5 includes a functional block diagram of an example implementation of the handheld diabetes management device;

FIG. 6 is a block diagram of an example implementation of firmware of the handheld diabetes management device;

FIG. 7 is a block diagram of an example implementation of a structured testing module of the handheld diabetes management device;

FIG. 8 is a block diagram of an example implementation of a sub-module of the structured testing module associated with executing a structured bG test; and

FIGS. 9 and 10 are flowcharts depicting example methods of performing structured bG tests on the handheld diabetes management device using only one or more of four firmware sub-routines for predetermined bG data collection procedures, respectively.

### DETAILED DESCRIPTION

The following description is merely illustrative in nature and is in no way intended to limit the disclosure, its application, or uses. For purposes of clarity, the same reference numbers will be used in the drawings to identify similar elements. As used herein, the phrase at least one of A, B, and C should be construed to mean a logical (A or B or C), using a non-exclusive logical or. It should be understood that steps within a method can be executed in different order without altering the principles of the present disclosure.

A handheld blood glucose (bG) management device includes a processor that executes firmware for operating the diabetes management device. The firmware is stored in firmware that is implemented in a non-modifiable portion of memory of the diabetes management device. The firmware can be thought of as a routine, portions of which can be executed by the processor to operate the diabetes management device.

One or more approvals of the firmware are typically necessary before the diabetes management device (executing the firmware) can be made publicly available. For example only, approval from one or more regulatory bodies (e.g., a Food and Drug Administration) can be required before the diabetes management device is made available in an area that is subject to the jurisdiction of the regulatory body. Before approving the firmware, a given regulatory body can require submission of a copy of the firmware, performance of one or more clinical tests to establish the operability of the firmware, and/or fulfillment of one or more other requirements.

Among other things, the firmware includes subroutines for executing each of four different types of bG data collection procedures. The four types of bG data collection procedures are: single sample bG data collection procedures; testing in pairs bG data collection procedures; time series bG data collection procedures; and X-point profile bG data collection procedures. X is an integer greater than 1 and may be, for example, 7 or 8. One or a combination of more than one of the bG data collection procedures can be prescribed to monitor/control any parameter related to a patient's diabetes care.

A user of the diabetes management device may be prompted to input a single blood sample for a single sample bG data collection procedure. The patient may be prompted to input a pair of blood samples for a testing in pairs bG data collection procedure. The patient may be prompted to input blood samples at predetermined intervals for a time series bG data collection procedure. The patient may be prompted to input X number of blood samples at approximately X predetermined times of a day for an X-point profile bG data collection procedure.

One or more different structured bG tests may be executed on the diabetes management device. Execution of a given structured bG test involves execution of one or more of the types of bG data collection procedures. Data for which one or more of the bG data collection procedures to execute for a given structured bG test is provided in a structured test module. Data for the order of execution of the one or more of the bG data collection procedures is also provided in the structured test module. Data for determining one or more parameters related to the diabetes management of a patient based on blood sample data provided for a given structured bG test is also provided in the structured test module.

The data of the structured test module could be written (hard coded) in the firmware in the firmware (in the non-modifiable portion of memory). In that case, however, the requirement of regulatory approval of the firmware may limit one's freedom to modify how the structured bG tests are executed if such modifications/updates are desired.

In the example diabetes management device of the present disclosure, the structured test module is stored in a modifiable portion of memory. In this manner, how many of the bG data collection procedures to execute for a given structured bG test, the order of execution of the one or more bG data collection procedures, and/or how the one or more parameters are calculated can be modified independently of the firmware. Because the firmware remains unchanged when data in the structured test module is modified, another approval of the firmware may be unnecessary. Not having to obtain another approval can provide significant and measurable cost savings (e.g., from not having to conduct another round of clinical testing). This can also enable modifications/updates to how structured bG tests are executed to be made publicly available sooner. Additionally, the same firmware can be distributed on diabetes management devices throughout the world and how the structured bG tests are performed can be updated, for example, to reflect local standards.

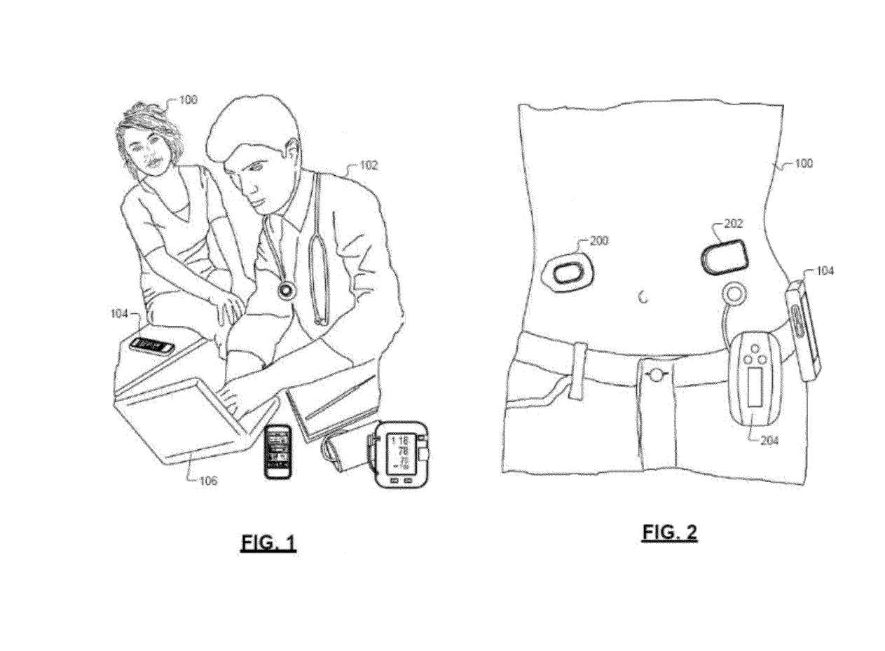

Referring now to FIG. 1, a patient 100 with diabetes and a health care professional 102 are shown in a clinical environment. The patient 100 with diabetes can be diagnosed with a metabolic syndrome, pre-diabetes, type 1 diabetes, type 2 diabetes, gestational diabetes, etc. Healthcare providers for diabetes are diverse and include nurses, nurse practitioners, physicians, endocrinologists, and others and are collectively referred to as health care professionals.

During a health care consultation, the patient 100 typically shares with the health care professional 102 a variety of data including blood glucose (bG) measurements, continuous glucose monitor data, amounts and type of insulin administered, amounts of food and beverages consumed, exercise schedules, health status, and other lifestyle information. The health care professional 102 can obtain additional data for the patient 100, such as measurements of HbA1C, cholesterol levels, plasma glucose, triglycerides, blood pressure, and weight. The data can be recorded manually or electronically on a handheld diabetes management device 104 (e.g., a handheld bG monitor device), a diabetes analysis software executed on a personal computer (PC) 106, and/or a web-based diabetes analysis site. The health care professional 102 can analyze the patient data manually or electronically using the diabetes analysis software and/or the web-based diabetes analysis site. After analyzing the data and reviewing how efficacious previously prescribed therapy is and how well the patient 100 followed the previously prescribed therapy, the health care professional 102 can decide whether to modify a therapy prescribed for the patient 100.

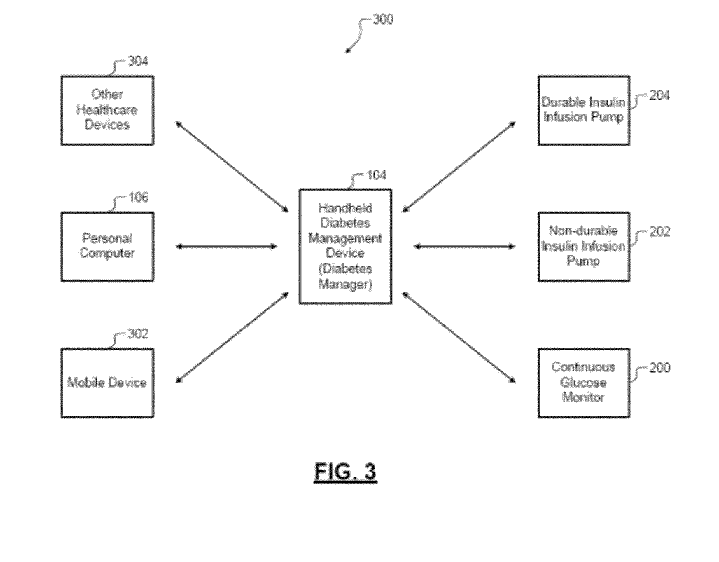

Referring now to FIG. 2, the patient 100 can use a continuous glucose monitor (CGM) 200, an ambulatory durable insulin infusion pump 204 or an ambulatory non-durable insulin infusion pump 202 (collectively insulin pump 202 or 204), and the diabetes management device 104. The CGM 200 can use a subcutaneous sensor to sense and monitor the amount of glucose (e.g., glucose concentration) of the patient 100. The CGM 200 communicates glucose measurements to the diabetes management device 104.

The diabetes management device 104 performs various tasks including measuring and recording bG measurements, determining an amount of insulin to be administered to the patient 100 via the insulin pump 202 or 204, receiving user input via a user interface, archiving data, performing structured bG tests, etc. The diabetes management device 104 can transmit instructions to the insulin pump 202 or 204, and the insulin pump 202 or 204 selectively delivers insulin to the patient 100. Insulin can be delivered in the form of a meal bolus dose, a correction bolus dose, a basal dose, etc.

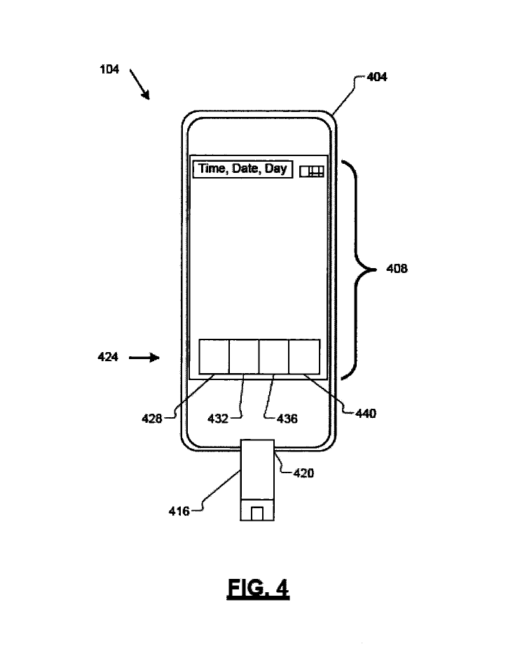

Referring now to FIG. 3, a diabetes management system 300 is shown which can be used by the patient 100 and/or the health care professional 102. The system 300 can include one or more of the following devices: the diabetes management device 104, the CGM 200, the insulin pump 202 or 204, a mobile device 302, the diabetes management software executed on the computer 106, and one or more other health care devices 304. The diabetes management device 104 can be configured as a system “hub” and communicate with one or more of the other devices of the system 300. The insulin pump 204, the mobile device 302, or another suitable device can alternatively serve as the system hub. Communication between various devices in the system 300 can be performed using wireless interfaces (e.g., Bluetooth) and/or wired interfaces (e.g., USB). Communication protocols used by these devices can include protocols compliant with the IEEE 11073 standard as extended using guidelines provided by Continua Health Alliance Design Guidelines. Further, health care records systems such as Microsoft HealthVault™ and Google Health™ can be used by the patient 100 and the health care professional 102 to exchange information.

The diabetes management software running on the computer 106 can include an analyzer-configurator that stores configuration information for devices of the system 300. For example only, the configurator has a database to store configuration information for the diabetes management device 104 and the other devices. A patient can interface the configurator through standard web based or computer graphical user interfaces (GUIs). The configurator selectively transmits patient-approved configurations to the devices of the system 300. The analyzer selectively retrieves data from the devices of the system 300, stores the data in a database, selectively analyzes the data, and outputs analysis results through standard web based or computer GUIs.

Referring now to FIG. 4, a high level illustration of an example embodiment of the diabetes management device 104 is presented. The diabetes management device 104 includes, among other things, a housing 404, user unit control switches (not specifically numbered), a touchscreen display 408, and a bG test strip port 420. The user unit control switches, for example, can include ON/OFF switches, volume switches, alarm switches for bG testing and/or insulin administration, and/or one or more other switches or other types of control devices that a patient can use to control functions/operations of the diabetes management device 104.

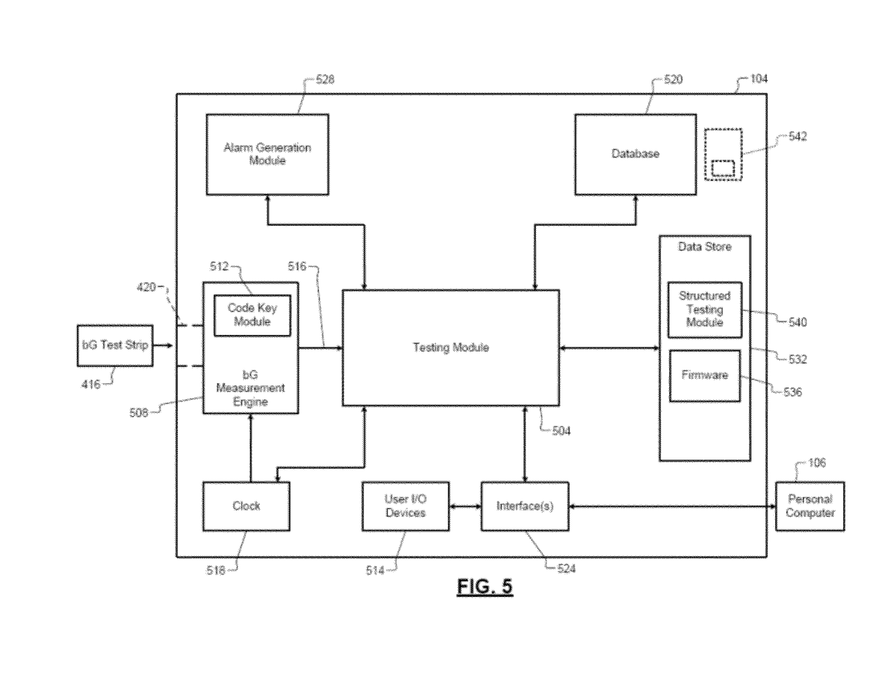

A bG test strip 416 can be inserted into the bG test strip port 420. The bG test strip 416 can be inserted into the bG test strip port 420 by a patient, from a test strip drum (not shown) located within the housing 404, or in another suitable manner. The bG test strip 416 is shown already inserted into the bG test strip port 420 in the example of FIG. 4 and not yet inserted into the bG test strip port 420 in the example of FIG. 5.

User selectable options 424 can be displayed on a portion of the display 408. The selectable options 424 can include a menu option 428, a bolus insulin option 432, a carbohydrate option 436, and an event option 440. One or more other user selectable options can additionally or alternatively be available. The patient can access a device menu for the diabetes management device 104 by selecting the menu option 428. The patient can input various insulin (and/or other medication) information (e.g., amount, insulin type, etc.) by selecting the bolus insulin option 432. The patient can input various carbohydrate intake information (e.g., amount) by selecting the carbohydrate option 436. The patient can also input other food intake information (e.g., protein content, fat content, etc.) by selecting the carbohydrate option 436. The patient can input various event related information (e.g., meals, exercise, periods of stress, etc.) that can affect the patient's bG measurements by selecting the event option 440.

Although the display 408 is described herein as a touchscreen display, the diabetes management device 104 can include another suitable form of display (e.g., LED, etc.). If a touchscreen display is not used, the user control switches can include specific buttons or controls by which the patient is able to select various options and input markers needed to select, input, and perform structured bG tests. Structured bG tests can also be referred to as focused tests.

The above description is a broad description of the diabetes management device 104. In practice, the diabetes management device 104 can include additional controls, input ports, output ports, etc., as can be desired to further enhance its utility or its use with other components and devices (e.g., computers, infusion pumps, cellular phones, etc.). The description of the diabetes management device 104 should not be taken as limiting as to the construction of the diabetes management device 104 or as to the features and capabilities of the diabetes management device 104.

As used herein, the term “module” can refer to, be part of, or include an Application Specific Integrated Circuit (ASIC); an electronic circuit; a combinational logic circuit; a field programmable gate array (FPGA); a processor (shared, dedicated, or group) that executes code; other suitable components that provide the described functionality; or a combination of some or all of the above, such as in a system-on-chip. The term “module” can include memory (shared, dedicated, or group) that stores code executed by the processor.

The term “code,” as used above, can include software, firmware, and/or microcode, and can refer to programs, routines, functions, classes, and/or objects. The term “shared,” as used above, means that some or all code from multiple modules can be executed using a single (shared) processor. In addition, some or all code from multiple modules can be stored by a single (shared) memory. The term “group,” as used above, means that some or all code from a single module can be executed using a group of processors. In addition, some or all code from a single module can be stored using a group of memories.

The apparatuses and methods described herein can be implemented by one or more computer programs executed by one or more processors. The computer programs include processor-executable instructions that are stored on a non-transitory, tangible, computer readable medium. The computer programs can also include stored data. Examples of the non-transitory, tangible, computer readable medium include, but are not limited to, nonvolatile memory, magnetic storage, and optical storage.

Referring now to FIG. 5, a functional block diagram of the diabetes management device 104 is presented. The diabetes management device 104 can include a testing module (e.g., a microprocessor based subsystem) 504 that can receive information from a bG measurement engine 508. The bG measurement engine 508 can be located adjacent the bG test strip port 420.

The bG measurement engine 508 reads (measures) a bG level of the bG test strip 416. The bG measurement engine 508 can include a code key module 512 that includes pre-calibrated data for determining a bG level from the bG test strip 416. The bG test strip 416 may be provided from the test strip drum housing unused bG test strips within the diabetes management device 104.

The bG measurement engine 508 generates bG sample data 516 based on its reading of the bG test strip 416. Among other things, the bG sample data 516 includes data indicative of the bG level of a blood sample on the bG test strip 416. The testing module 504 can also receive bG sample data from other sources, such as via the CGM 200, the display 408, and/or another suitable source. The testing module 504 can receive user input data via one or more user input/output (I/O) devices 514, such as the display 408, one or more buttons/switches/etc., and/or one or more other user I/O devices.

The bG measurement engine 508 can also generate the bG sample data 516 to indicate the date and time when the bG test strip 416 was read. In other words, the bG measurement engine 508 can include a time stamp with the bG sample data 516. In various implementations, the testing module 504 can selectively time stamp the bG sample data 516 and can time stamp user input data and other data when it is received.

A clock 518 can provide the date and time. The patient can configure the present date and time, and the clock 518 thereafter tracks the present date and time. In various implementations, the present date and time can be acquired from (e.g., synchronized with) the computer 106. The bG measurement engine 508 communicates the bG sample data 516 to the testing module 504.

The testing module 504 is in communication with a database 520 used to store bG sample data, user input data, and other data. For example only, the testing module 504 can store each piece of bG sample data received in the database 520. The testing module 504 is also in communication with the display 408 and one or more interfaces 524. Each of the interfaces 524 can provide an interface between the diabetes management device 104 and an external device, such as the computer 106, the insulin pump 202 or 204, the CGM 200, the mobile device 302, the other health care devices 304, and/or one or more other suitable external devices.

The testing module 504 is also in communication with an alarm generation module 528. The alarm generation module 528 can generate one or more alarms when prompted by the testing module 504. For example only, the alarm generation module 528 can generate audible, tactile (e.g., vibratory), and/or visual alarms. The alarms can be used, for example, in prompting the patient to input data for a structured bG test. The testing module 504 is also in communication with a data store 532. For example only, the data store 532 can be Not-And (NAND) type flash memory and/or another suitable type of memory. While shown alternatively in the example of FIG. 5, the database 520 can be implemented within the data store 532 in various implementations.

The data store 532 includes firmware 536 and a structured testing module 540 as shown in the example of FIG. 5. The data store 532 can also include one or more other modules (not shown).

Firmware for operating the diabetes management device 104 is stored in the firmware 536. In the case of the data store 532 being flash memory or another type of re-writable tangible storage medium, the firmware 536 can be within a partition that is made non-modifiable (e.g., marked as read only). In this manner, the firmware cannot be updated, re-written, or otherwise modified via user input to the diabetes management device 104. To update the firmware, the partition would have to be changed to remove the non-modifiable status of the firmware via base input/output system (BIOS) of the diabetes management device 104. In various implementations, the firmware 536 can be implemented independently of the data store 532, such as within read only memory (ROM) or another suitable type of memory as shown in dashed lines at 542. In various implementations, the firmware 536 may be stored on a removable storage device, such as a thumb/jump drive, a code key, removable memory, and/or another suitable type of removable storage device. In all implementations, however, the firmware 536 is or is made non-modifiable. In contrast, data stored in the structured testing module 540 is modifiable. For example only, the structured testing module 540 may be implemented in an area of the data store 532 marked as being readable/writable.

Portions of the firmware are selectively executed to operate the diabetes management device 104. For example only, the testing module 504 selectively executes one or more portions of the firmware to execute a structured bG test. To execute a structured bG test, the testing module 504 executes one or more of four predetermined types of bG data collection procedures as specified in the structured testing module 540. The routines for executing the four predetermined types of bG data collection procedures is stored in the firmware 536.

A structured bG test may be executed to estimate one or more parameters related to diabetes care of the patient. For example only, the parameter may be an amount of long acting and/or basal insulin presently carried by the patient (which can be referred to as insulin on board), an insulin to carbohydrate ratio for the patient, an insulin sensitivity of the patient, and/or a time over which bolus insulin is active in the patient (i.e., an insulin acting time), and/or one or more other parameters related to the diabetes care of the patient. A structured bG test may be initiated, for example, at a predetermined date and time. The predetermined date and time may be set, for example, by the patient, a health care professional, by the diabetes management device 104, or in another suitable manner.

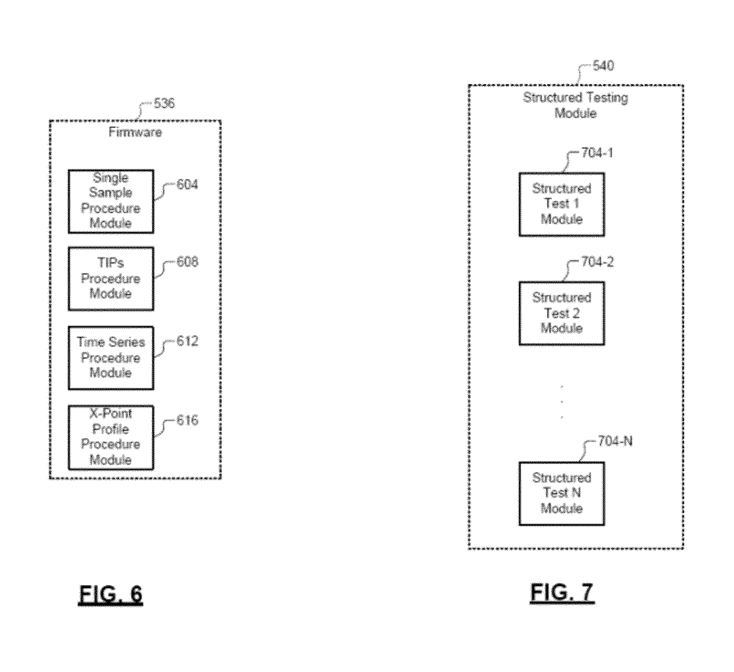

FIG. 6 is an example block diagram of the firmware 536. With continuing reference to FIG. 5, the routines for executing the four predetermined types of bG data collection procedures can be thought of as respective sub-modules of the firmware 536.

A first predetermined type of bG data collection procedure can be referred to as a single sample procedure, and a second predetermined type of bG data collection procedure can be referred to as a testing in pairs (TIPs) procedure. A third predetermined type of bG data collection procedure can be referred to as a time series procedure, and a fourth predetermined type of bG data collection procedure can be referred to as an X-point profile procedure. The routines for executing the first, second, third, and fourth predetermined types of bG data collection procedures are stored in a single sample procedure module 604, a TIPs procedure module 608, a time series procedure module 612, and an X-point profile procedure module 616, respectively. The single sample procedure module 604, the TIPs procedure module 608, the time series procedure module 612, and the X-point profile procedure module 616 can each be referred to as a sub-routine of the firmware 536.

The predetermined types of bG data collection procedures involve prompting the patient to input one or more bG samples according to predetermined schedules, respectively. Each of the predetermined schedules is different. The predetermined schedule for a single sample procedure generally involves prompting the patient to input a single bG sample (e.g., via a bG test strip 20) at approximately a predetermined time. The predetermined time may be, for example, pre-breakfast (also referred to as fasting). The predetermined schedule for a TIPs procedure generally involves prompting the patient to input a pair of bG samples around (e.g., one before and one after) one or more events. The event(s) may be, for example, consumption of a meal, exercise, administration of medication, a psychological event, or another suitable type of event that may affect the patient's bG level. The predetermined schedule for a time series procedure generally involves prompting the patient to input bG samples at predetermined intervals (e.g., once per hour). The predetermined schedule for an X-point profile procedure generally involves prompting the patient to input X number of bG samples at approximately X predetermined times during a one day period (e.g., 12:00 A.M. to 11:59 P.M.). X is an integer greater than 3. For example only, X may be equal to 7 or 8 in various implementations. In implementations where X is equal to 7, the predetermined times may be: 1) a pre-breakfast time; 2) a post-breakfast time; 3) a pre-lunch time; 4) a post-lunch time; 5) a pre-dinner time; 6) a post-dinner time; and 7) a bedtime time. In implementations where X is equal to 8, the predetermined times may include the seven predetermined times plus an eighth predetermined time between the predetermined times for bedtime and pre-breakfast. For example only, the eighth predetermined time may be approximately 3:00 a.m. The predetermined times may be patient chosen times, health care professional chosen times, default times, and/or other suitably chosen times. The testing module 504 may limit the number of structured bG tests that are being performed on the diabetes management device 104 at a given time to one. The predetermined types of bG data collection procedures also involve assessing whether associated entry, adherence, and/or exit criteria are satisfied. Entry, adherence, and exit criteria are discussed further below.

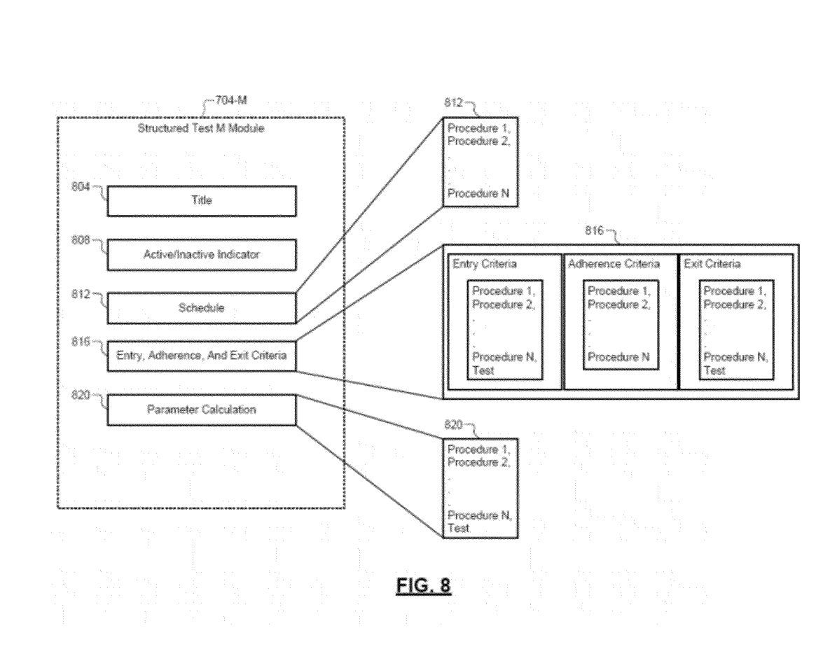

FIG. 7 is an example block diagram of the structured testing module 540. With continuing reference to FIGS. 5-6, the diabetes management device 104 is capable of executing N number of structured bG tests. The data associated with a given structured bG test is stored in a structured testing module. For example only, the data associated with N number of structured bG tests is stored in structured test modules 704-1, 704-2, . . . , 704-N (collectively referred to as structured test modules 704), respectively. N is an integer greater than 1. N may be set to a predetermined number by default by the manufacturer of the diabetes management device 104.

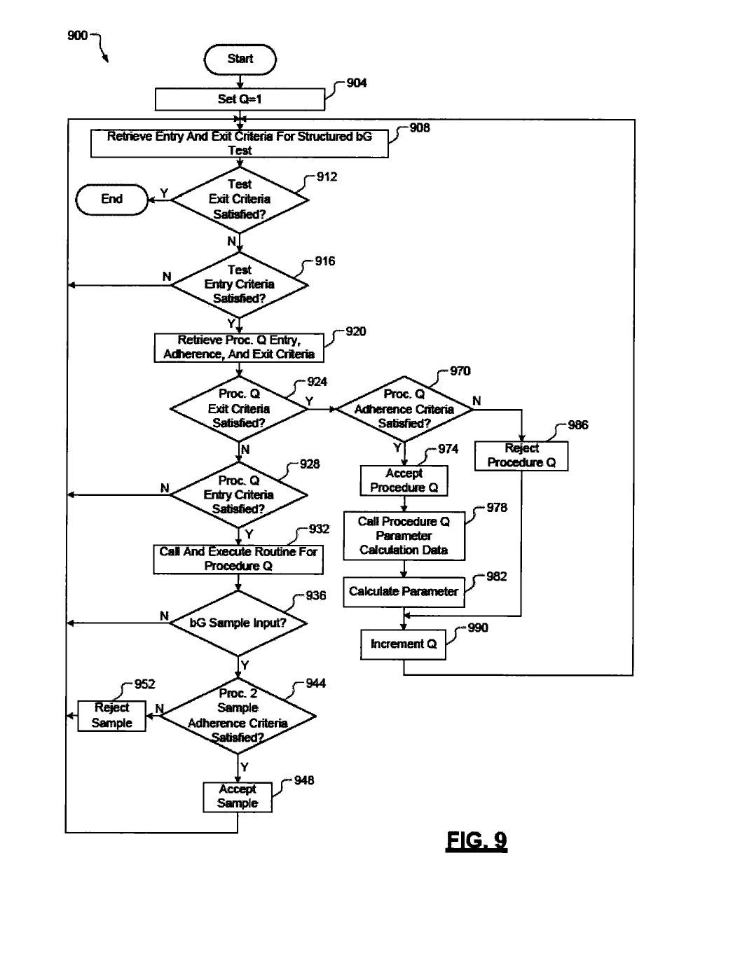

FIG. 8 is an example block diagram of a structured test module 704-M associated with a given one of the structured bG tests. M is an integer greater than zero and less than or equal to N. The discussion of FIG. 8 in conjunction with the structured test module 704-M is for example only and may also be applicable to the other structured test modules 704.

Referring also to FIGS. 5-7, the structured test module 704-M may include title data 804 and an active/inactive indicator 808. The active/inactive indicator 808 indicates whether the structured bG test associated with the structured test module 704-M is available for execution. A structured test graphical user interface (GUI) (not shown) may be displayed via the display 408 based on inputs to the diabetes management device 104.

The testing module 504 may selectively display structured bG tests for selection and execution via the structured test GUI. More specifically, the testing module 504 may display the title data 804 via the structured test GUI when the active/inactive indicator 808 is in an active state. If the active/inactive indicator 808 is in an inactive state, testing module 504 may omit the title data 804 from the structured test GUI. The patient may initiate execution of the structured bG test via the structured test GUI.

Additionally or alternatively, a second structured test GUI (not shown) may be displayed via the software executed on the computer 106 based on inputs to the computer 106. The title data associated with each of the structured test modules 704 may be displayed via the structured test GUI. The structured test modules 704 may be modified via the software executed on the computer 106.

As stated above, each of the structured bG tests involves executing one or more of the predetermined types of bG data collection procedures (via executing the respective modules in the firmware 536). The structured test module 704-M includes a routine for executing the structured bG test including the one or more bG data collection procedures and for determining one or more parameters related to diabetes care based on data input during execution of the one or more bG data collection procedures. The structured test module 704-M also includes data that is called and used in executing the structured bG test and the one or more bG data collection procedures, such as various entry, adherence, and exit criteria.

For example only, the structured test module 704-M includes execution data 812. The execution data 812 may include a routine for selectively calling for execution of the one or more bG data collection procedures (i.e., the modules 604-616) and for determining one or more parameters associated with diabetes care of the patient 100.

The structured test module 704-M includes adherence, entry, and exit criteria data 816 that is selectively called for during execution of the one or more bG data collection procedures. The adherence, entry, and exit criteria data 816 include entry and exit criteria for the structured bG test. The adherence, entry, and exit criteria data 816 may further include entry, adherence, and exit criteria for each of the bG data collection procedures to be executed for the structured bG test.

For example only, the entry criteria for the structured bG test include one or more criterion used in determining whether to begin performing the structured bG test. For example only, the entry criteria for the structured bG test can include a time (and/or date) after when the structured bG test is to begin. The entry criteria for the structured bG test can additionally or alternatively include a threshold age of the patient, a threshold range for HbA1c of the patient, a threshold length of time that the patient has had diabetes, a specified type of diabetes diagnosed to the patient, a threshold body mass index (BMI) of the patient, and/or a threshold fasting plasma glucose level. The entry criteria for the structured bG test can additionally or alternatively include one or more other suitable criteria. Alternatively, the entry criteria for the structured bG test can instead be entry criteria for one or more of the one or more bG data collection procedures to be executed for the structured bG test.

The exit criteria for the structured bG test include one or more criterion used in determining whether to end execution of the structured bG test. For example only, the exit criteria for the structured bG test can include an ending date (and/or time) for the structured bG test. The exit criteria for the structured bG test can additionally or alternatively include a threshold value (e.g., of a counter) that is indicative of when the one or more bG data collection procedures are complete. The counter (not shown) may be incremented each time that one of the one or more bG data collection procedures is complete such that when the counter is greater than the threshold value, each of the one or more bG data collections procedures are complete. Alternatively, the exit criteria for the structured bG test can instead be exit criteria for the last one of the one or more bG data collection procedures to be executed for the structured bG test.

The entry criteria for a given one of the bG data collection procedures include one or more criterion used in determining whether to begin executing the one of the bG data collection procedures. For example only, the entry criteria can include a time (and/or a date) to begin executing the one of the bG data collection procedures. The entry criteria for a given one of the bG data collection procedures can additionally or alternatively include one or more other suitable criteria, such as user input data, whether user input data has been received, whether user input data includes predetermined characteristics, and/or whether one or more other ones of the bG data collection procedures have been completed.

The adherence criteria for the bG samples input for a given one of the bG data collection procedures include one or more criterion used in determining whether to accept or reject a bG sample input during execution of the one of the bG data collection procedures. This type of adherence criteria may be referred to as sample adherence criteria. The sample adherence criteria can be used in determining whether the bG sample is acceptable for consideration in making a medical determination (e.g., determining a parameter related to the diabetes care of the patient) associated with the one of the bG data collection procedures. For example only, if a bG sample is expected on a given date between time X and time Y, the adherence criteria for the bG sample can include the date and a window of time defined by time X and time Y. Accepted bG samples can be marked for consideration in making the medical determination. Rejected bG samples can be used in determining whether exit criteria for the one of the bG data collection procedures and/or the structured bG test are satisfied.

The testing module 504 selectively accepts or rejects received bG samples based on comparisons of one or more characteristics of the bG sample data with the associated adherence criteria. For example only, the testing module 504 can mark a received bG sample as accepted when the associated adherence criteria are satisfied. Conversely, the testing module 504 can mark the bG sample as rejected when the bG sample does not satisfy the adherence criteria.

The adherence criteria for a given one of the bG data collection procedures include one or more criterion used in determining whether to accept or reject all of the bG sample data collected during execution of the one of the bG data collection procedures. This type of adherence criteria may be referred to as procedure adherence criteria. The procedure adherence criteria may be used in determining whether bG sample data collected for the one of the bG data collection procedures, as a whole, is acceptable for consideration in making the medical determination. For example only, the procedure adherence criteria for a given one of the bG data collection procedures can include a threshold number of bG samples satisfying the adherence criteria. For example only, if X bG samples are expected to be input for a given one of the bG data collection procedures and Y of the X bG samples are expected to be accepted, Y can be adherence criteria for the one of the bG data collection procedures. X and Y are integers, and Y is greater than zero and less than or equal to X. The adherence criteria for a given one of the bG data collection procedures can additionally or alternatively include one or more other suitable criteria.

The exit criteria for a given one of the bG data collection procedures include one or more criterion used in determining whether to end execution of the one of the bG data collection procedures. For example only, the exit criteria for a given one of the bG data collection procedures can include an ending date and time for the one of the bG data collection procedures. For example only, if all of the bG samples for a given one of the bG data collection procedures are expected to be input before a given date, the exit criteria for the one of the bG data collection procedures can include the given date.

The exit criteria for the one of the bG data collection procedures can additionally or alternatively include a threshold number of missed or rejected bG samples. For example only, if X bG samples are expected for the one of the bG data collection procedures and Y of the X bG samples is a maximum number of the expected bG samples that can be missed or rejected, Y can be exit criteria for the one of the bG data collection procedures. X and Y are integers, and Y is greater than or equal to zero and less than or equal to X. The exit criteria for a given one of the bG data collection procedures can additionally or alternatively include one or more other suitable criteria. For example only, the exit criteria for a given one of the bG data collection procedures can include whether one or more of the bG data collection procedures was executed before the given one of the bG data collection procedures and/or whether the adherence criteria for one or more of those one or more bG data collection procedures was/were satisfied.

Further information on adherence, exit, and entry criteria can be found in paragraphs [0048] and [0092]-[0116] of commonly assigned U.S. patent application Ser. No. 12/643,338, filed Dec. 21, 2009, (Pub No. 2010-0212675) and titled “Structured Testing Method for Diagnostic or Therapy Support of a Patient with a Chronic Disease and Devices Thereof.” Further information on adherence, exit, and entry criteria can also be found in paragraphs [0087]-[0112] of commonly assigned U.S. patent application Ser. No. 12/643,415, filed on Dec. 21, 2009, (Pub. No. 2010-0218132) and titled “Management Method and System for Implementation, Execution, Data Collection, and Data Analysis of a Structured Collection Procedure Which Runs on a Collection Device.” The above patent applications, including the above mentioned paragraphs, are incorporated by reference in their entirety.

The structured test module 704-M may also include parameter calculation data 820. The testing module 504 may determine a result of one or more of the one or more bG data collection procedures using the parameter calculation data 820. The parameter calculation data 820 may include parameter calculation data for each of the one or more bG data collection procedures. The testing module 504 may determine a result of a given one of the bG data collection procedures executed for the structured bG test using the bG samples input for the one of the bG data collection procedures that satisfied the adherence criteria. The parameter calculation data 820 may include one or more functions and/or mappings that relate the bG sample data to the result. For example only, the parameter calculation data 820 may include one set of functions and/or mappings for each of the one or more bG data collection procedures. The result may be a parameter related to diabetes care of the patient or a result that may be used with one or more other parameters to determine a parameter related to the diabetes care of the patient. For example only, the bG level of accepted blood samples and/or one or more other suitable values may be used to determine the patient's insulin on board, the patient's insulin to carbohydrate ratio, the patient's insulin sensitivity, the patient's insulin acting time, and/or one or more other parameters related to the diabetes care of the patient.

The testing module 504 may determine one or more parameters related to the diabetes care of the patient based on one or more results of the one or more bG data collection procedures executed pursuant to the schedule data 812. The parameter calculation data 820 may include one or more functions and/or mappings that relate the results of the one or more bG data collection procedures executed to the one or more parameters that are related to the diabetes care of the patient.

A HCP and/or the patient may configure various parameters of the structured test module 704-M via a structured testing GUI presented via the computer 106 and/or the diabetes management device 104. For example only, a title may be input for the structured test associated with the structured test module 704-M via the structured testing GUI and the title data 804 may be generated and stored accordingly. The active/inactive indicator 808 may be set via the structured testing GUI. The order of execution of the one or more bG data collection procedures can be selected and set via the structured testing GUI. The schedule data 812 can be generated and stored based on the selected order of execution. The entry and exit criteria for the structured bG test and the entry, adherence, and exit criteria for each of the one or more bG data collection procedures to be executed can be specified via the structured testing GUI. The entry, adherence, and exit criteria data 816 can be generated and stored accordingly. How the one or more parameters related to the diabetes care of the patient are to be determined can be specified via the structured testing GUI. The parameter calculation data 820 can be generated and stored accordingly. In various implementations, an HCP and a patient may be able to configure parameters of the structured test module 704-M to different degrees. For example only, the patient may not be authorized to configure the entry, adherence, and exit criteria data 816 while the HDP can. The HCP and the patient are both unable to configure the firmware 536 (including the firmware for the bG data collection procedures).

Unlike the firmware 536, the data stored in the structured testing module 540 is modifiable and updatable. For example only, one or more entry, adherence, and/or exit criteria can be remotely updated or modified via the software executed on the computer 106 or in another suitable manner. This is advantageous as it allows the ready modification of such criteria should such modification be or become preferable. An additional significant advantage is that since the firmware is not modified, a future modification of entry, adherence, and/or exit criteria might not trigger the need to perform additional verification testing of the software (e.g., for regulatory approval).

Another significant advantage is that the execution of the structured bG test can be modified without changing the firmware. For example only, how many of the bG data collection procedures are executed for a given structured bG test, the order of execution of the one or more bG data collection procedures for the given structured bG test, and/or how one or more results are determined for the given structured bG test can be modified. These characteristics can be updated or modified via the software executed on the computer 106 or in another suitable manner. Because how many of the bG data collection procedures are executed for the given structured bG test, the order of execution of the one or more bG data collection procedures for the given structured bG test, and how one or more results are determined can be modified (via modifying the structured test module 704 associated with the structured bG test) without changing the firmware, another round of verification testing may not be necessary. Such changes may be made based on, for example, the desires of a health care professional, locally accepted medical practices, etc.

Another significant benefit is that the availability of one or more structured bG tests for execution can be modified. For example only, the active/inactive indicator 808 of one or more of the structured test modules 704 can be modified via the software executed on the computer 106 or in another suitable manner. The ability to change which one or more structured bG tests are available for execution by the patient may, for example, help ensure that a non-prescribed structured bG test is not executed by the patient accidentally or otherwise.

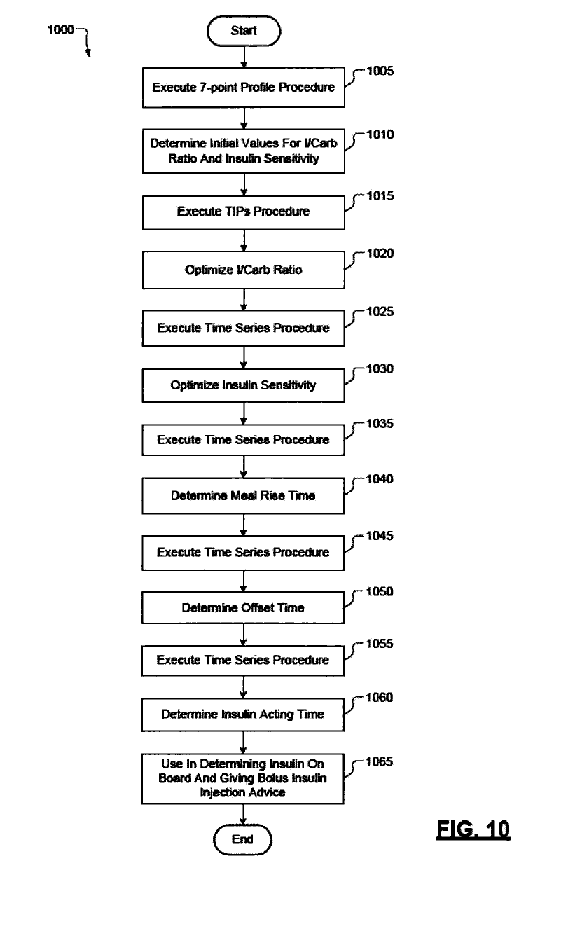

FIG. 9 is a flowchart depicting an example method 900 of executing a structured bG test including one or more of the bG data collection procedures. Control may begin at 904 where control sets a value (Q) equal to 1 or another suitable initialization value. As described below, Q may be incremented each time that one of the one or more bG data collection procedures to be executed for the structured bG test is completed. In this manner, Q indicates which one of the one or more bG data collection procedures to execute according to the order of execution. For example only, Q being equal to 1 indicates that the first one of the one or more bG data collection procedures in the order of execution should be executed.

Control may retrieve the entry and exit criteria for the structured bG test at 908. When to retrieve the entry and exit criteria for the structured bG test and where the entry and exit criteria are stored may be located in the schedule data. At 912, control determines whether the exit criteria for the structured bG test are satisfied. If true, control may end; if false, control may continue with 916. For example only, one of the exit criteria may be a predetermined value (X), where X is an integer greater than zero or one. X corresponds to the total number of the one or more bG data collection procedures to be executed for the structured bG test. The exit criteria for the structured bG test may be deemed satisfied when Q is greater than or equal to X.

Control determines whether the entry criteria for the structured bG test are satisfied at 916. If true, control continues with 920; if false, control may return to 908. Control reads Q at 920, and control may retrieve the entry, adherence, and exit criteria for the Q-th one of the one or more bG data collection procedures from the structured testing module 540 at 920. For example only, when Q is equal to 1, control may retrieve the entry, adherence, and exit criteria for the first one of the one or more bG data collection procedures in the order of execution. Retrieval of the entry, adherence, and exit criteria may be called for by the module of the firmware 536 associated with the bG data collection procedure or by the schedule data.

At 924, control determines whether the exit criteria for the Q-th one of the one or more bG data collection procedures are satisfied. If true, control may continue with 970, which is discussed further below; if false, control may continue with 928. Control may determine whether the entry criteria for the Q-th one of the one or more bG data collection procedures are satisfied at 928. If true, control may call and execute the routine for the Q-th one of the one or more bG data collection procedures stored in the firmware 536 at 932. The routine for the Q-th one of the one or more bG data collection procedures is stored in the single sample procedure module 604, the TIPs procedure module 608, the time series procedure module 612, or the X-point profile procedure module 616 in the firmware 536.

Control determines whether a bG sample has been input at 936. If true, may continue with 944; if false, control may return to 908. Control determines whether the sample adherence criteria are satisfied at 944. If true, control may mark the input bG sample as accepted at 948 and return to 908; if false, control may mark the input bG sample as rejected at 952 and return to 908.

Referring back to 970 (i.e., when the exit criteria for the Q-th one of the one or more bG data collection procedures are satisfied at 924), control may determine whether the adherence criteria for the Q-th one of the one or more bG data collection procedures are also satisfied. In other words, control may determine whether the procedure adherence criteria for the Q-th one of the one or more bG data collection procedures are satisfied at 970. If true, control may mark the Q-th one of the one or more bG data collection procedures as accepted at 974, call the parameter calculation data associated with the Q-th one of the one or more bG data collection procedures at 978, and calculate a parameter associated with diabetes management of the patient 100 based on accepted bG samples of the Q-th one of the one or more bG data collection procedures and the parameter calculation data at 982. If false, control may mark the Q-th one of the one or more bG data collection procedures as rejected at 986. Control may increment Q at 990 after 982 or 986 and return to 908. In this manner, if the exit criteria for the structured bG test are not satisfied (e.g., Q is not greater than or equal to X in 912), control may begin executing a next one of the one or more bG data collection procedures in the order of execution.

FIG. 10 is flowchart depicting an example method 1000 of executing a structured bG test according to schedule data stored in the one of the structured test modules 704 associated with the structured bG test. The example method 1000 of FIG. 10 may be executed, for example, for advice giving regarding administration of bolus insulin.

Control may begin with 1005 where control executes an X-point profile procedure using the X-point profile procedure module 616 of the firmware 536. X may be equal to 7 or 8 in various implementations, and X may be specified in the structured test module 704 associated with the structured bG test. The predetermined times for the X expected bG samples may also be specified in the structured test module 704 associated with the structured bG test. Control may use entry, adherence, and exit criteria stored in the structured test module 704 associated with the structured bG test in executing the X-point profile procedure. A number of consecutive days during which the X-point profile procedure should be executed may also be stored in the structured test module 704 associated with the structured bG test. Control may execute the X-point profile procedure each day for the number of consecutive days. For example only, the number may be approximately 3. Control may determine initial values of the patient's insulin to carbohydrate (I/Carb) ratio and the patient's insulin sensitivity at 1010 using accepted bG samples input during the X-point profile procedure and parameter calculation data stored in the structured test module 704 associated with the structured bG test.

At 1015, control may execute a first TIPs procedure using the TIPs procedure module 608 of the firmware 536. Control may use entry, adherence, and exit criteria associated with the first TIPs procedure that is stored in the structured test module 704. For example only, an entry criterion for the first TIPs procedure may be that a pre-meal bG level of the patient is within a predetermined range. When to expect the bG samples for the first TIPs procedure are stored in the structured test module 704 associated with the structured bG test. For example only, a first bG sample may be expected before a meal and a second bG sample may be expected after the meal or before a second (e.g., next or later) meal. Control may optimize the patient's I/Carb ratio at 1020. Control may optimize the patient's I/Carb ratio using accepted bG sample data input during the first TIPs procedure and parameter calculation data associated with the first TIPs procedure stored in the structured test module 704.

Control may execute a first time series procedure at 1025 using the time series procedure module 612 of the firmware 536. Control may use entry, adherence, and exit criteria associated with the first time series procedure that is stored in the structured test module 704. For example only, an entry criterion for the first time series procedure may be that a pre-meal bG level of the patient is greater than an upper limit of a predetermined target range. The first time series procedure includes prompting the patient to input bG samples at predetermined intervals for a predetermined period beginning after an offset period. The predetermined intervals, the predetermined period, and the offset period associated with the first time series procedure are stored in the structured test module 704. For example only, the predetermined intervals may be approximately 1 hour, the predetermined period may be approximately 5 hours, and the offset period may be approximately zero. Control may optimize the patient's insulin sensitivity at 1030. Control may optimize the patient's insulin sensitivity using accepted bG sample data input during the time series procedure and parameter calculation data associated with the first time series procedure stored in the structured test module 704.

At 1035, control may execute a second time series procedure using the time series procedure module 612 of the firmware 536. Control may use entry, adherence, and exit criteria associated with the second time series procedure that is stored in the structured test module 704. For example only, an entry criterion for the second time series procedure may be consumption of a meal. The second time series procedure includes prompting the patient to input bG samples at predetermined intervals for a predetermined period beginning after an offset period. The predetermined intervals, the predetermined period, and the offset period associated with the second time series procedure are stored in the structured test module 704. For example only, the predetermined intervals may be approximately 30 minutes, the predetermined period may be approximately 3-6 hours, and the offset period may be approximately 15 minutes.

Alternatively or additionally, control may execute a second TIPs procedure at 1035 using the TIPs procedure module 608 of the firmware 536. Control may use entry, adherence, and exit criteria associated with the second TIPs procedure that is stored in the structured test module 704. The second TIPs procedure may include prompting the patient to input a first bG sample before a meal and to input a second bG sample after the meal. When to prompt the patient to input the bG samples for the second TIPs procedure is stored in the structured test module 704.

Control may determine a meal rise (expected increase in bG after a meal) for the patient at 1040. Control may determine the meal rise using accepted bG sample data input during the second time series (and/or TIPs) procedure(s). Control may determine the meal rise further using parameter calculation data associated with the second time series and/or TIPs procedures stored in the structured test module 704.

Control may execute a third time series procedure at 1045 using the time series procedure module 612 of the firmware 536. Control may use entry, adherence, and exit criteria associated with the third time series procedure stored in the structured test module 704. The third time series procedure includes prompting the patient to input bG samples at predetermined intervals for a predetermined period beginning after an offset period. The predetermined intervals, the predetermined period, and the offset period associated with the third time series procedure are stored in the structured test module 704. For example only, the predetermined intervals may be approximately 15 minutes, the predetermined period may be approximately 2 hours, and the offset period may be approximately zero. Control may additionally determine an offset time for the patient at 1050. Control may determine the offset time using accepted bG sample data input during the third time series procedure and parameter calculation data associated with the third time series procedure stored in the structured test module 704.

At 1055, control may execute a fourth time series procedure using the time series procedure module 612 of the firmware 536. Control may use entry, adherence, and exit criteria associated with the fourth time series procedure stored in the structured test module 704. The fourth time series procedure includes prompting the patient to input bG samples at predetermined intervals for a predetermined period beginning after an offset period. The predetermined intervals, the predetermined period, and the offset period associated with the fourth time series procedure are stored in the structured test module 704. For example only, the predetermined intervals may be approximately 1 hour, the predetermined period may be approximately 3 to 6 hours, and the offset period may be approximately zero. Control may determine an insulin acting time for the patient at 1060. Control may determine the insulin acting time using accepted bG sample data input during the fourth time series procedure and parameter calculation data associated with the fourth time series procedure stored in the structured test module 704.

In various implementations, control may execute two or more of the predetermined bG data collection procedures concurrently. For example only, control may execute the first TIPs procedure of 1015 and the first time series procedures of 1025 concurrently before optimizing the I/Carb ratio and the insulin sensitivity at 1020 and 1030. For another example only, control may execute the second time series (and/or TIPs) procedure of 1035, the third time series procedure of 1045, and the fourth time series procedure of 1055 concurrently before determining the meal rise, the offset time, and the insulin acting time. Control may also further optimize the I/Carb ratio and/or the insulin sensitivity based on one or more of the meal rise, the offset time, and the insulin acting time.

Control may use one or more of the I/Carb ratio, the insulin sensitivity, the meal rise, the offset time, and the insulin acting time in determining the insulin on board value for the patient at 1065. Control may determine the insulin on board value, for example, using firmware in the firmware 536 with one or more of the I/Carb ratio, the insulin sensitivity, the meal rise, the offset time, and the insulin acting time as inputs. Control may selectively use the insulin on board determined at a given time for one or more reasons, such as in giving the patient advice regarding a bolus insulin injection amount at the given time.

Each of the other structured bG tests executable by the diabetes management device 104 executes one or more of the bG data collection procedures. For example only, one or more time series procedures may be executed for a meal characterization structured bG test. For another example only, one or more time series procedures may be executed for a basal insulin adjustment structured bG test used in conjunction with an insulin pump. For another example only, one or more time series procedures, one or more TIPs procedures, and/or one or more X-point profile procedures may be executed for an adaptive bolus insulin structured bG test used in conjunction with a CGM. For another example only, one or more time series procedures may be executed for an HbA1C predictive structured bG test. For another example only, one or more time series procedures, one or more TIPs procedures, and/or one or more X-point profile procedures may be executed for a basal insulin adjustment structured bG test used when the patient skips a meal. For another example only, one or more single sample procedures, one or more time series procedures, and/or one or more TIPs procedures may be executed for a complete insulin titration therapy structured bG test. Other structured bG tests can also be executed via execution of one or a combination of more than one of the 4 predetermined bG data collection procedures.

A handheld diabetes management device with predetermined types of blood glucose (bG) data collection procedures for improved structured testing, the handheld diabetes management device includes a bG measurement engine, a data store, a display, and a testing module. The bG measurement engine selectively measures bG levels in blood samples of a patient and that generates sample data indicative of the bG levels, respectively. The data store stores a first bG data collection procedure that prompts the patient to input at least one blood sample according to a first predetermined routine, a second bG data collection procedure that prompts the patient to input at least one blood sample according to a second predetermined routine that is different from the first predetermined routine, and a plurality of structured tests. Each of the structured tests is executable for determining a parameter related to diabetes care of the patient and has data for executing one or more of the first and second bG data collection procedures. Each of the first and second bG data collection procedures is accessible to each of the structured tests for execution. The testing module is in communication with the bG measurement engine, the display, and the memory. The testing module selectively executes one of the structured tests, including one or more of the first and second bG data collection procedures.

In other features, the data store further stores a third bG data collection procedure that prompts the patient to input at least one blood sample according to a third predetermined routine that is different than the second predetermined routine and a fourth bG data collection procedure that prompts the patient to input at least one blood sample according to a fourth predetermined routine that is different than the third predetermined routine. Each of the structured tests has data for executing one or more of the first, second, third, and fourth bG data collection procedures. Each of the third and fourth bG data collection procedures is accessible to each of the structured tests for execution.

In still other features, the first and second predetermined routines are stored in a non-modifiable portion of the data store.

In further features, the data store includes flash memory, and the first and second predetermined routines are stored in a portion of the flash memory that is marked as read-only.

In still further features, the data store includes read only memory (ROM), and the first and second predetermined routines are stored in the ROM.

In other features, an order of execution for the one or more of the first and second bG data collection procedures for the structured tests is stored in the data store and is modifiable. The testing module executes the one or more of the first and second bG data collection procedures according to the order of execution.

In still other features, one or more entry criterion, one or more adherence criterion, and one or more exit criterion are stored in the data store and are modifiable. The testing module selectively executes the one or more of the first and second bG data collection procedures based on the one or more entry criterion, the one or more adherence criterion, and the one or more exit criterion.

In further features, parameter calculation data for the one of the structured tests is stored in the data store and is modifiable. The testing module selectively determines the parameter related to the diabetes care of the patient using the calculation data and sample data input for the one of the structured tests.

In still further features, the calculation data includes at least one of a function and a mapping that relates the sample data to the parameter related to diabetes care of the patient.

A handheld diabetes management device with predetermined types of blood glucose (bG) data collection procedures for improved structured testing, the handheld diabetes management device includes a data store, a display, and a testing module. The data store has data for executing a plurality of structured tests, each of the structured tests calling for execution of one or more of: a first bG data collection procedure including prompting the patient to input a first blood sample at a first predetermined time; a second bG data collection procedure including prompting the patient to input second and third blood samples at second and third predetermined times, respectively; a third bG data collection procedure including prompting the patient to input M blood samples at predetermined intervals beginning after a predetermined offset period, wherein M is an integer greater than 2; and a fourth bG data collection procedure including prompting the patient to input N number of blood samples at N predetermined times of a day, respectively, wherein N is an integer greater than 6. The testing module that is in communication with the display and the data store and that selectively executes one of the structured tests including the one or more of the first, second, third, and fourth bG data collection procedures.

In other features, the first, second, third, and fourth routines for executing the first, second, third, and fourth bG data collection procedures are stored in a non-modifiable portion of the data store.

In still other features, the data store includes flash memory, and the first, second, third, and fourth routines are stored in a portion of the flash memory that is marked as read-only.

In further features, the data store includes read only memory (ROM), and the first, second, third, and fourth routines are stored in the ROM.

In still further features, an order of execution for the one or more of the first, second, third, and fourth bG data collection procedures is stored in the data store and is modifiable, and the testing module selectively executes the one or more of the first, second, third, and fourth bG data collection procedures according to the order of execution.

In other features, the order of execution is modifiable to add and to subtract one or more of the first, second, third, and fourth bG data collection procedures from the order of execution.

In still other features, one or more entry criterion, one or more adherence criterion, and one or more exit criterion are stored in the data store and are modifiable, and the testing module selectively executes the one or more of the first, second, third, and fourth bG data collection procedures based on the one or more entry criterion, the one or more adherence criterion, and the one or more exit criterion.

In further features, parameter calculation data for the one of the structured tests is stored in the data store and is modifiable, and the testing module selectively determines a parameter related to diabetes care of the patient using the calculation data and sample data input for the one of the structured tests.

In still further features, the calculation data includes at least one of a function and a mapping that relates the sample data to the parameter related to diabetes care of the patient.

In other features, the testing module selectively executes one of the structured tests including the two or more of the first, second, third, and fourth bG data collection procedures.

A computer readable storage medium of a handheld diabetes management device that includes a blood glucose (bG) measurement engine that measures a bG level of a blood sample and that further includes a touch screen display, the computer readable storage medium includes a non-modifiable portion and a modifiable portion. The non-modifiable portion includes firmware for operating the handheld diabetes management device and includes: a first routine executable for prompting the patient to input a first blood sample at a first predetermined time; a second routine executable for prompting the patient to input second and third blood samples at second and third predetermined times, respectively; a third routine executable for prompting the patient to input M blood samples at predetermined intervals beginning after a predetermined offset period, wherein M is an integer greater than 2; and a fourth routine executable for prompting the patient to input N number of blood samples at N predetermined times of a day, respectively, wherein N is an integer greater than 6. The modifiable portion includes data for executing one of a plurality of structured tests including: an order of execution of one or more of the first, second, third, and fourth routines for the one of the structured tests; entry, adherence, and exit criteria for each of the one or more of the first, second, third, and fourth routines; and at least one of a function and a mapping for determining a parameter related to diabetes care of the patient based on blood samples input pursuant to the one or more of the first, second, third, and fourth routines.

The broad teachings of the disclosure can be implemented in a variety of forms. Therefore, while this disclosure includes particular examples, the true scope of the disclosure should not be so limited since other modifications will become apparent to the skilled practitioner upon a study of the drawings, the specification, and the following claims.

What is claimed is:

1. A handheld diabetes management device with predetermined types of blood glucose (bG) data collection procedures for improved structured testing, the handheld diabetes management device comprising:
a bG measurement engine that selectively measures bG levels in blood samples of a patient and that generates sample data indicative of the bG levels, respectively;
a data store for storing a first bG data collection procedure that prompts the patient to input at least one blood sample according to a first predetermined routine, a second bG data collection procedure that prompts the patient to input at least one blood sample according to a second predetermined routine that is different from the first predetermined routine, and a plurality of structured tests,
each of the structured tests executable for determining a parameter related to diabetes care of the patient and having data for executing one or more of the first and second bG data collection procedures, and
each of the first and second bG data collection procedures being accessible to each of the structured tests for execution;
a display; and
a testing module that is in communication with the bG measurement engine, the display, and the memory and that selectively executes one of the structured tests, including one or more of the first and second bG data collection procedures.

2. The handheld diabetes management device of claim 1 wherein the data store further stores a third bG data collection procedure that prompts the patient to input at least one blood sample according to a third predetermined routine that is different than the second predetermined routine and a fourth bG data collection procedure that prompts the patient to input at least one blood sample according to a fourth predetermined routine that is different than the third predetermined routine,
each of the structured tests has data for executing one or more of the first, second, third, and fourth bG data collection procedures, and
each of the third and fourth bG data collection procedures being accessible to each of the structured tests for execution.

3. The handheld diabetes management device of claim 1 wherein the first and second predetermined routines are stored in a non-modifiable portion of the data store.

4. The handheld diabetes management device of claim 3 wherein the data store includes flash memory, and
wherein the first and second predetermined routines are stored in a portion of the flash memory that is marked as read-only.

5. The handheld diabetes management device of claim 3 wherein the data store includes read only memory (ROM), and
wherein the first and second predetermined routines are stored in the ROM.

6. The handheld diabetes management device of claim 3 wherein an order of execution for the one or more of the first and second bG data collection procedures for the structured tests is stored in the data store and is modifiable, and
wherein the testing module executes the one or more of the first and second bG data collection procedures according to the order of execution.

7. The handheld diabetes management device of claim 3 wherein one or more entry criterion, one or more adherence criterion, and one or more exit criterion are stored in the data store and are modifiable, and
wherein the testing module selectively executes the one or more of the first and second bG data collection procedures based on the one or more entry criterion, the one or more adherence criterion, and the one or more exit criterion.

8. The handheld diabetes management device of claim 3 wherein parameter calculation data for the one of the structured tests is stored in the data store and is modifiable, and
wherein the testing module selectively determines the parameter related to the diabetes care of the patient using the calculation data and sample data input for the one of the structured tests.

9. The handheld diabetes management device of claim 8 wherein the calculation data includes at least one of a function and a mapping that relates the sample data to the parameter related to diabetes care of the patient.

10. A handheld diabetes management device with predetermined types of blood glucose (bG) data collection procedures for improved structured testing, the handheld diabetes management device comprising:
a data store with data for executing a plurality of structured tests, each of the structured tests calling for execution of one or more of:
a first bG data collection procedure including prompting the patient to input a first blood sample at a first predetermined time;
a second bG data collection procedure including prompting the patient to input second and third blood samples at second and third predetermined times, respectively;
a third bG data collection procedure including prompting the patient to input M blood samples at predetermined intervals beginning after a predetermined offset period, wherein M is an integer greater than 2; and
a fourth bG data collection procedure including prompting the patient to input N number of blood samples at N predetermined times of a day, respectively, wherein N is an integer greater than 6, and
a display; and
a testing module that is in communication with the display and the data store and that selectively executes one of the structured tests including the one or more of the first, second, third, and fourth bG data collection procedures.

11. The handheld diabetes management device of claim 10 wherein first, second, third, and fourth routines for executing the first, second, third, and fourth bG data collection procedures are stored in a non-modifiable portion of the data store.

12. The handheld diabetes management device of claim 11 wherein the data store includes flash memory, and
wherein the first, second, third, and fourth routines are stored in a portion of the flash memory that is marked as read-only.

13. The handheld diabetes management device of claim 11 wherein the data store includes read only memory (ROM), and
wherein the first, second, third, and fourth routines are stored in the ROM.

14. The handheld diabetes management device of claim 11 wherein an order of execution for the one or more of the first, second, third, and fourth bG data collection procedures is stored in the data store and is modifiable, and
wherein the testing module selectively executes the one or more of the first, second, third, and fourth bG data collection procedures according to the order of execution.

15. The handheld diabetes management device of claim 14 wherein the order of execution is modifiable to add and to subtract one or more of the first, second, third, and fourth bG data collection procedures from the order of execution.

16. The handheld diabetes management device of claim 11 wherein one or more entry criterion, one or more adherence criterion, and one or more exit criterion are stored in the data store and are modifiable, and
wherein the testing module selectively executes the one or more of the first, second, third, and fourth bG data collection procedures based on the one or more entry criterion, the one or more adherence criterion, and the one or more exit criterion.

17. The handheld diabetes management device of claim 11 wherein parameter calculation data for the one of the structured tests is stored in the data store and is modifiable, and
wherein the testing module selectively determines a parameter related to diabetes care of the patient using the calculation data and sample data input for the one of the structured tests.

18. The handheld diabetes management device of claim 17 wherein the calculation data includes at least one of a function and a mapping that relates the sample data to the parameter related to diabetes care of the patient.

19. The handheld diabetes management device of claim 11 wherein the testing module selectively executes one of the structured tests including the two or more of the first, second, third, and fourth bG data collection procedures.

20. A computer readable storage medium of a handheld diabetes management device that includes a blood glucose (bG) measurement engine that measures a bG level of a blood sample and that further includes a touch screen display, the computer readable storage medium comprising:
a non-modifiable portion that includes firmware for operating the handheld diabetes management device and that includes:
a first routine executable for prompting the patient to input a first blood sample at a first predetermined time;
a second routine executable for prompting the patient to input second and third blood samples at second and third predetermined times, respectively;
a third routine executable for prompting the patient to input M blood samples at predetermined intervals beginning after a predetermined offset period, wherein M is an integer greater than 2; and
a fourth routine executable for prompting the patient to input N number of blood samples at N predetermined times of a day, respectively, wherein N is an integer greater than 6; and
a modifiable portion that includes data for executing one of a plurality of structured tests including:
an order of execution of one or more of the first, second, third, and fourth routines for the one of the structured tests;
entry, adherence, and exit criteria for each of the one or more of the first, second, third, and fourth routines; and
at least one of a function and a mapping for determining a parameter related to diabetes care of the patient based on blood samples input pursuant to the one or more of the first, second, third, and fourth routines.
